(19) United States
(12) Patent Application Publication
(10) Pub. No.: US 2025/0127460 A1
(43) Pub. Date: Apr. 24, 2025

(54) PREDICTION DEVICE, METHOD OF GENERATING PREDICTION MODEL, AND COMPUTING DEVICE

(71) Applicant: NIHON KOHDEN CORPORATION, Tokyo (JP)

(72) Inventors: Takuya KAWASHIMA, Tokorozawa-shi (JP); Daisuke HORIGUCHI, Tokorozawa-shi (JP)

(73) Assignee: NIHON KOHDEN CORPORATION, Tokyo (JP)

(21) Appl. No.: 18/828,369

(22) Filed: Sep. 9, 2024

(30) Foreign Application Priority Data
Oct. 18, 2023 (JP) 2023-179591

(51) Int. Cl. A61B 5/00

(52) U.S. Cl. CPC A61B 5/7275; A61B 5/7264

(57) ABSTRACT

An interface receives time series data including multiple observed values of an observed parameter that are acquired at different time points for obtaining physiological information of a subject. A processor inputs the time series data into a prediction model to perform prediction of one or more unobserved values of the observed parameter. The processor causes an output device to visualize a range within which the one or more unobserved values may fall. The range is changed in accordance with a time interval between the different time points.

### CROSS-REFERENCE TO RELATED APPLICATION

The present application is based on Japanese Patent Application No. 2023-179591 filed on Oct. 18, 2023, the entire contents of which are incorporated herein by reference.

### BACKGROUND

The presently disclosed subject matter relates to a prediction device configured to input, to a prediction model, time series data including multiple observed values of an observed parameter acquired from a subject for obtaining physiological information of the subject, thereby performing prediction about the physiological information. The presently disclosed subject matter also relates to a method of generating the prediction model as well as a computing device configured to generate the prediction model.

Prediction models that are generated with machine learning techniques to replace or support clinical decisions of medical workers in the field of medical clinical practice are known. For example, Non-patent Document 1 discloses a model adapted to predict specific physiological information based on time series data of an observed parameter acquired from a living body. Examples of specific physiological information include the future value of the observed parameter (used as a time series forecasting model), the in-hospital mortality probability (used as a classification model), and the remaining stay time of the intensive care unit (ICU) (used as a regression model). As described in, for example, Non-patent Document 2, the prediction accuracy of the prediction model may be visualized as a confidence interval.

Non-patent Document 1: G. Harerimana, et al. “A Multi-Headed Transformer Approach for Predicting the Patient's Clinical Time-Series Variables From Charted Vital Signs”, IEEE Access (Volume 10), 105993-106004, Oct. 3, 2022

Non-patent Document 2: Y. Du, et al., “Time-series and Machine Learning Scenarios for COVID-19 Infection Prediction”, The 2nd International Conference on Biological Engineering and Medical Science, 397-403, Jun. 7, 2022

### SUMMARY

It is demanded to provide a user interface capable of enabling a user to visually recognize changes in the prediction accuracy of physiological information that may occur in accordance with changes in observation environment of an observed parameter for obtaining physiological information of a subject.

A first illustrative aspect of the presently disclosed subject matter provides a prediction device, comprising:
an interface configured to receive time series data including multiple observed values of an observed parameter that are acquired at different time points for obtaining physiological information of a subject; and
a processor configured to:
   input the time series data into a prediction model to perform prediction of one or more unobserved values of the observed parameter; and
   cause an output device to visualize a range within which the one or more unobserved values may fall,
wherein the range is changed in accordance with a time interval between the different time points.

A second illustrative aspect of the presently disclosed subject matter provides a method of generating, with a computing device, the prediction model according to the first illustrative aspect, comprising:
acquiring a first observation data set including multiple observed values of the observed parameter acquired from a living body with a first time interval;
acquiring a second observation data set including multiple observed values of the observed parameter acquired from the living body with a second time interval that is different from the first time interval;
storing, in association with the first time interval, a difference between an observed value of the observed parameter acquired subsequently to the first observation data set and an unobserved value of the observed parameter forecasted on the basis of the first observation data set;
storing, in association with the second time interval, a difference between an observed value of the observed parameter acquired subsequently to the second observation data set and an unobserved value of the observed parameter forecasted on the basis of the second observation data set; and
performing machine learning of the prediction model so as to enable prediction of a range within which one or more unobserved values of the observed parameter may fall on the basis of a time interval of multiple observed values of the observed parameter acquired from a subject.

A third illustrative aspect of the presently disclosed subject matter provides a computing device configured to generate the prediction model according to the first illustrative aspect, comprising:
an interface configured to receive:
   a first observation data set including multiple observed values of the observed parameter acquired from a living body with a first time interval; and
   a second observation data set including multiple observed values of the observed parameter acquired from the living body with a second time interval that is different from the first time interval; and
a processor configured to:
   store, in association with the first time interval, a difference between an observed value of the observed parameter acquired subsequently to the first observation data set and an unobserved value of the observed parameter forecasted on the basis of the first observation data set;
   store, in association with the second time interval, a difference between an observed value of the observed parameter acquired subsequently to the second observation data set and an unobserved value of the observed parameter forecasted on the basis of the second observation data set; and
   perform machine learning of the prediction model so as to enable prediction of a range within which one or more unobserved values of the observed parameter may fall on the basis of a time interval of multiple observed values of the observed parameter acquired from a subject.

In general, the shorter the time interval of acquired multiple observed values included in the time series data of the observed parameter, the higher the prediction accuracy of the unobserved value of the observed parameter that is forecasted by the prediction model. On the other hand, for example, the axillary temperature is typically measured manually by a medical worker in a clinical practice. Here, there may be a case where it is changed (not constant) the time interval for acquiring multiple observed values of a certain observed parameter. In other words, there may be a case where the prediction accuracy of the unobserved value of the observed parameter as forecasted dynamically changes. However, according to the configuration of each of the first to third illustrative aspects, not only it is possible to visualize a range within which an unobserved value of the observed parameter predicted in response to the inputted time series data of the observed parameter may fall, but also it is possible to dynamically change the range in accordance with the time interval of the acquired observed values. Accordingly, it is possible to provide a user interface that enables the user to visually recognize a change in the forecasting accuracy of the unobserved value of the observed parameter that may occur in accordance with a change in the observation environment of the observed parameter.

A fourth illustrative aspect of the presently disclosed subject matter provides a prediction device, comprising:
an interface configured to receive time series data including multiple observed values of an observed parameter that are acquired at different time points for obtaining physiological information of a subject; and
a processor configured to:
   input the time series data into a prediction model to perform prediction of one or more occurrence probabilities of an event related to the observed parameter; and
   cause an output device to visualize a range within which the one or more occurrent probabilities may fall,
wherein the range is changed in accordance with a time interval between the different time points.

A fifth illustrative aspect of the presently disclosed subject matter provides a method of generating, with a computing device, the prediction model according to the fourth illustrative aspect, comprising:
acquiring a first observation data set including multiple observed values of the observed parameter acquired from a living body with a first time interval;
acquiring a second observation data set including multiple observed values of the observed parameter acquired from the living body with a second time interval that is different from the first time interval;
storing, in association with the first time interval, a difference between a value corresponding to whether or not an event related to the observed parameter is actually occurred with or subsequently to acquisition of the first observation data set and a value corresponding to an occurrence probability of the event estimated on the basis of the first observation data set;
storing, in association with the first time interval, a difference between a value corresponding to whether or not the event is actually occurred with or subsequently to acquisition of the second observation data set and a value corresponding to an occurrence probability of the event estimated on the basis of the second observation data set; and
performing machine learning of the prediction model so as to enable prediction of a range within which one or more occurrence probabilities of the event may fall on the basis of a time interval of multiple observed values of the observed parameter acquired from a subject.

A sixth illustrative aspect of the presently disclosed subject matter provides a computing device configured to generate the prediction model according to the fourth illustrative aspect, comprising:
an interface configured to receive:
   a first observation data set including multiple observed values of the observed parameter acquired from a living body with a first time interval; and
   a second observation data set including multiple observed values of the observed parameter acquired from the living body with a second time interval that is different from the first time interval; and
a processor configured to:
   store, in association with the first time interval, a difference between a value corresponding to whether or not an event related to the observed parameter is actually occurred with or subsequently to acquisition of the first observation data set and a value corresponding to an occurrence probability of the event estimated on the basis of the first observation data set;
   store, in association with the first time interval, a difference between a value corresponding to whether or not the event is actually occurred with or subsequently to acquisition of the second observation data set and a value corresponding to an occurrence probability of the event estimated on the basis of the second observation data set; and
   perform machine learning of the prediction model so as to enable prediction of a range within which one or more occurrence probabilities of the event may fall on the basis of a time interval of multiple observed values of the observed parameter acquired from a subject.

In general, the shorter the time interval of acquired multiple observed values included in the time series data of the observed parameter, the higher the occurrence probability of an event related to the observed parameter that is estimated by the prediction model. On the other hand, for example, the axillary temperature is typically measured manually by a medical worker in a clinical practice. Here, there may be a case where it is changed (not constant) the time interval for acquiring multiple observed values of a certain observed parameter. In other words, there may be a case where the prediction accuracy of the occurrence probability of the event as estimated dynamically changes. However, according to the configuration of each of the fourth to sixth illustrative aspects, not only it is possible to visualize a range within which an occurrence probability of an event related to the observed parameter predicted in response to the inputted time series data of the observed parameter may fall, but also it is possible to dynamically change the range in accordance with the time interval of the acquired observed values. Accordingly, it is possible to provide a user interface that enables the user to visually recognize a change in the estimation accuracy of the occurrence probability of the event related to the observed parameter that may occur in accordance with a change in the observation environment of the observed parameter.

A seventh illustrative aspect of the presently disclosed subject matter provides a prediction device, comprising:
an interface configured to receive time series data including multiple observed values of a first observed parameter that are acquired at different time points for obtaining physiological information of a subject; and
a processor configured to:
   input the time series data into a prediction model to perform prediction of one or more observed values of a second observed parameter that is different from the first observed parameter; and
   cause an output device to visualize a range within which the one or more observed values of the second observed parameter may fall,
wherein the range is changed in accordance with a time interval between the different time points.

An eighth illustrative aspect of the presently disclosed subject matter provides a method of generating, with a computing device, the prediction model according to the seventh illustrative aspect, comprising:
acquiring a first observation data set including multiple observed values of the first observed parameter acquired from a living body with a first time interval;
acquiring a second observation data set including multiple observed values of the first observed parameter acquired from the living body with a second time interval that is different from the first time interval;
storing, in association with the first time interval, a difference between an observed value of the second observed parameter acquired subsequently to the first observation data set and an observed value of the second observed parameter estimated on the basis of the first observation data set;
storing, in association with the second time interval, a difference between an observed value of the second observed parameter acquired subsequently to the second observation data set and an observed value of the second observed parameter estimated on the basis of the second observation data set; and
performing machine learning of the prediction model so as to enable prediction of a range within which one or more observed values of the second observed parameter may fall on the basis of a time interval of multiple observed values of the first observed parameter acquired from a subject.

A ninth illustrative aspect of the presently disclosed subject matter provides a computing device configured to generate the prediction model according to the seventh illustrative aspect, comprising:
an interface configured to receive:
   a first observation data set including multiple observed values of the first observed parameter acquired from a living body with a first time interval; and
   a second observation data set including multiple observed values of the first observed parameter acquired from the living body with a second time interval that is different from the first time interval; and
a processor configured to:
   store, in association with the first time interval, a difference between an observed value of the second observed parameter acquired subsequently to the first observation data set and an observed value of the second observed parameter estimated on the basis of the first observation data set;
   store, in association with the second time interval, a difference between an observed value of the second observed parameter acquired subsequently to the second observation data set and an observed value of the second observed parameter estimated on the basis of the second observation data set; and
   perform machine learning of the prediction model so as to enable prediction of a range within which one or more observed values of the second observed parameter may fall on the basis of a time interval of multiple observed values of the first observed parameter acquired from a subject.

In general, the shorter the time interval of acquired multiple observed values included in the time series data of the observed parameter, the higher the prediction accuracy of the observed value of another observed parameter that is estimated by the prediction model. On the other hand, for example, the axillary temperature is typically measured manually by a medical worker in a clinical practice. Here, there may be a case where it is changed (not constant) the time interval for acquiring multiple observed values of a certain observed parameter. In other words, there may be a case where the prediction accuracy of the observed value of another observed parameter as estimated dynamically changes. However, according to the configuration of each of the seventh to ninth illustrative aspects, not only it is possible to visualize a range within which an observed value of another observed parameter predicted in response to the inputted time series data of the observed parameter may fall, but also it is possible to dynamically change the range in accordance with the time interval of the acquired observed values of the observed parameter. Accordingly, it is possible to provide a user interface that enables the user to visually recognize a change in the forecasting accuracy of the observed value of another observed parameter that may occur in accordance with a change in the observation environment of the observed parameter.

### DESCRIPTION OF THE DRAWINGS

FIG. 1 illustrates a functional configuration of a prediction system according to one exemplary embodiment.

FIG. 2 illustrates an exemplary flow of processing executed in a prediction device of FIG. 1.

FIG. 3 illustrates an exemplary operation of the prediction device of FIG. 1.

FIG. 4 illustrates another exemplary operation of the prediction device of FIG. 1.

FIG. 5 illustrates another exemplary operation of the prediction device of FIG. 1.

FIG. 6 illustrates another exemplary operation of the prediction device of FIG. 1.

FIG. 7 illustrates another exemplary operation of the prediction device of FIG. 1.

### DESCRIPTION OF EMBODIMENTS

Exemplary embodiments will be described in detail with reference to the accompanying drawings. In the drawings, the scale is appropriately changed in order to make each element to be described have a recognizable size.

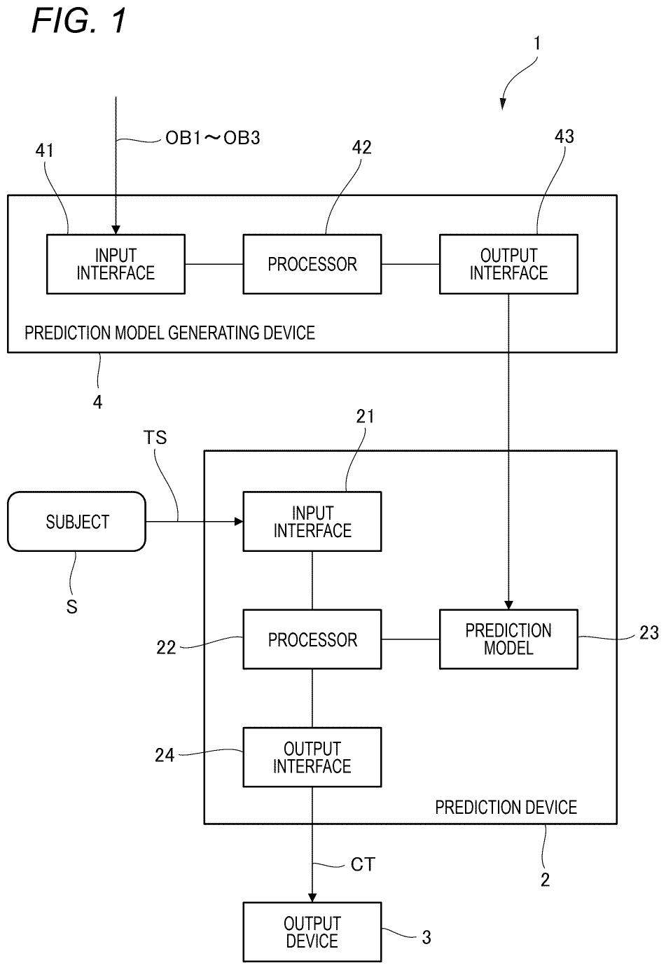

FIG. 1 illustrates a functional configuration of a prediction system 1 according to an exemplary embodiment. The prediction system 1 includes a prediction device 2.

The prediction device 2 according to the present example is a device configured to predict, based on time series data TS of axillary temperature values acquired from a subject S at or before a certain time point, a value of the axillary temperature after the time point (in the future). The value of axillary temperature is an example of an observed parameter. Examples of other observed parameters include a respiratory rate that is obtained by visually observing thoracic motions, a Glasgow Coma Scale (GCS) that is obtained by visually confirming the state of consciousness, a urine volume that is measured in a urine collection bag, an index value that is obtained through a blood test (the number of erythrocytes, the number of leukocytes, a blood lactate value, an arterial oxygen partial pressure, and the like), and a blood pressure that is obtained by non-invasive measurement. A future value of the observed parameter is an example of the physiological information.

As used herein, the term “time series of an observed parameter” means change with time of a value of an observed parameter that is acquired at multiple time points. The interval between the multiple time points may be constant or may not be constant.

The prediction device 2 includes an input interface 21. The input interface 21 is configured as a hardware interface that can receive the above-described time series data TS. The time series data TS may be inputted from an adequate sensor or measurement device attached to the subject S, or may be inputted from an adequate input device and a user operation. The time series data TS may be in the form of analog data or digital data, in accordance with the specification of the input source. In the case where the time series data TS is in the form of analog data, the input interface 21 includes an adequate conversion circuit including an A/D converter.

The prediction device 2 includes a processor 22 and a prediction model 23. The prediction model 23 is a computer algorithm adapted to be executed by the processor 22. The prediction model 23 is configured to output, as a prediction result, a range within which an unobserved value of axillary temperature of the subject S in the future may fall, with respect to the time series data TS as an input.

The prediction system 1 includes an output device 3. Examples of the output device 3 include a display device, a printing device, and the like. The prediction device 2 includes an output interface 24. The processor 22 is configured to input the time series data TS received by the input interface 21 to the prediction model 23, and configured to output, from the output interface 24, control data CT for causing the output device 3 to visualize the prediction result outputted from the prediction model 23.

The output interface 24 is configured as a hardware interface adapted to output the control data CT. The control data CT may be in the form of analog data or digital data, in accordance with the specification of the output device 3. In the case where the control data CT is in the form of analog data, the output interface 24 includes an adequate conversion circuit including a D/A converter.

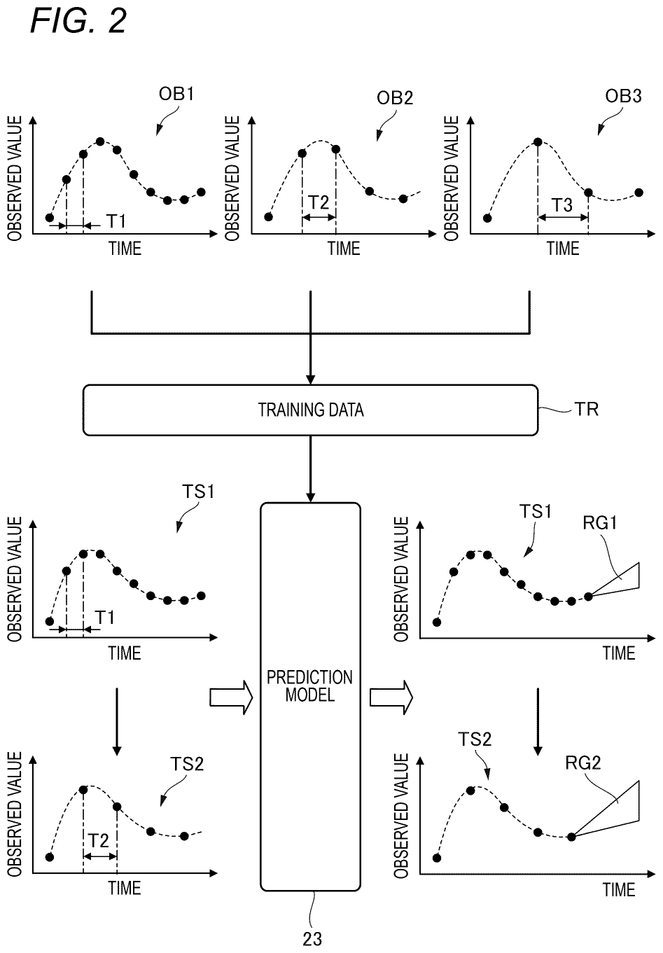

FIG. 2 illustrates first time series data TS1 including multiple observed values of the axillary temperature acquired from the subject S with a first time interval T1. By inputting the first time series data TS1 to the prediction model 23, a first range RG1 within which an unobserved value of the axillary temperature subsequent to the first time series data TS1 may fall is predicted to be subjected to the visualization by the output device 3.

The prediction device 2 according to this exemplary embodiment is configured to change a range within which an unobserved value of the axillary temperature that is to be visualized by the output device 3 may fall, in accordance with a time interval between the multiple observed values included in the time series data TS received by the input interface 21.

In second time series data TS2 illustrated in FIG. 2, multiple observed values of the axillary temperature are acquired with a second time interval T2. The second time interval T2 is longer than the first time interval T1. The second time series data TS2 may be acquired from the same subject as the first time series data TS1, or may be acquired from another subject.

By inputting the second time series data TS2 to the prediction model 23, a second range RG2 within which an unobserved value of the axillary temperature subsequent to the second time series data TS2 may fall is predicted to be subjected to the visualization by the output device 3. The second range RG2 is larger than the first range RG1.

As illustrated in FIG. 1, the prediction system 1 includes a prediction model generating device 4. The prediction model 23 of the prediction device 2 is generated through machine learning that is performed by the prediction model generating device 4. The prediction model generating device 4 includes an input interface 41, a processor 42, and an output interface 43. The prediction model generating device 4 is an example of a computing device.

With reference to FIG. 2, a method of generating, with the prediction model generating device 4, a prediction model 23 capable of realizing the above-described operation will be described.

The input interface 41 receives a first observation data set OB1 including multiple observed values of the axillary temperature acquired from a living body with a first time interval T1.

The processor 42 is configured to, based on multiple observed values of the axillary temperature acquired from the living body with the first time interval T1, forecast an observed value of the axillary temperature acquired subsequently to the observed values.

The processor 42 stores, in association with the first time interval T1, a difference between the observed value of the axillary temperature acquired subsequently to the first observation data set OB1 and the unobserved value of the axillary temperature forecasted on the basis of the first observation data set OB1. The stored information forms a part of training data TR.

Similarly, the input interface 41 receives a second observation data set OB2 including multiple observed values of the axillary temperature acquired from the living body with the second time interval T2. The second time interval T2 is different from the first time interval T1. In this example, the second time interval T2 is longer than the first time interval T1.

The processor 42 performs interpolation such that the time interval T2 of the second observation data set OB2 is resampled with the first time interval T1. In addition, the processor 42 calculates a difference between an observed value of the axillary temperature acquired subsequently to the second observation data set OB2 and the unobserved value of the axillary temperature forecasted on the basis of the resampled second observation data set OB2. The processor 42 then stores the difference in association with the second time interval T2. The stored information forms a part of training data TR.

Moreover, the input interface 41 receives a third observation data set OB3 including multiple observed values of the axillary temperature acquired from the living body with a third time interval T3. The third time interval T3 is different from each of the first time interval T1 and the second time interval T2. In this example, the third time interval T3 is longer than the second time interval T2.

The processor 42 performs interpolation such that the time interval T3 of the third observation data set OB3 is resampled with the first time interval T1. In addition, the processor 42 calculates a difference between an observed value of the axillary temperature acquired subsequently to the third observation data set OB3 and the unobserved value of the axillary temperature forecasted on the basis of the resampled third observation data set OB3. The processor 42 stores the difference in association with the third time interval T3. The stored information forms a part of training data TR.

The same processing may be performed on another observation data set including observed values of the axillary temperature acquired with a different time interval to generate the training data TR.

The processor 42 executes machine learning of the prediction model 23 with the training data TR generated as described above. Specifically, the neural network is caused to learn the influence of the time interval of multiple observed values of the axillary temperature acquired from the subject on the forecasting accuracy of the unobserved value of the axillary temperature. As a result, the prediction model 23 is configured to predict a range within which an unobserved value of the axillary temperature may fall, with the time interval of the multiple observed values included in the inputted time series data TS of the axillary temperature as the feature.

In general, the shorter the time interval of acquired multiple observed values included in the time series data TS of the axillary temperature, the higher the prediction accuracy of the unobserved value of the axillary temperature that is forecasted by the prediction model 23. On the other hand, there may be a case where it is changed (not constant) the time interval for acquiring multiple observed values of a certain observed parameter like the axillary temperature. In other words, there may be a case where the prediction accuracy of the unobserved value of the axillary temperature as forecasted dynamically changes. However, according to the configuration of this exemplary embodiment, not only it is possible to visualize a range within which an unobserved value of the axillary temperature predicted in response to the inputted time series data TS of the axillary temperature may fall, but also it is possible to dynamically change the range in accordance with the time interval of the acquired observed values. Accordingly, it is possible to provide a user interface that enables the user to visually recognize a change in the forecasting accuracy of the unobserved value of the axillary temperature that may occur in accordance with a change in the observation environment of the axillary temperature.

It should be noted that the expression “a range within which a value may fall” as used herein broadly means “a difference between a value as predicted and a value as actually observed”. The statistical standard error of the difference, the difference between the maximum value and the minimum value of the difference, the quartile of the difference, the percentile of the difference, the confidence interval of the difference, and the standard deviation of the difference may be included in the “a range within which a value may fall”.

In this example, an observation data set including multiple observed values with a larger time interval may be generated by downsampling a portion of the observation data set including multiple observed values with a smaller time interval. For example, at least one of the second observation data set OB2 and the third observation data set OB3 may be generated by downsampling some of the multiple observed values included in the first observation data set OB1.

According to such a configuration, it is possible to generate a prediction model capable of being adapted to a case where the time interval between the multiple observed values included in the time series data TS is changed, efficiently from a data set including a limited number of observed values.

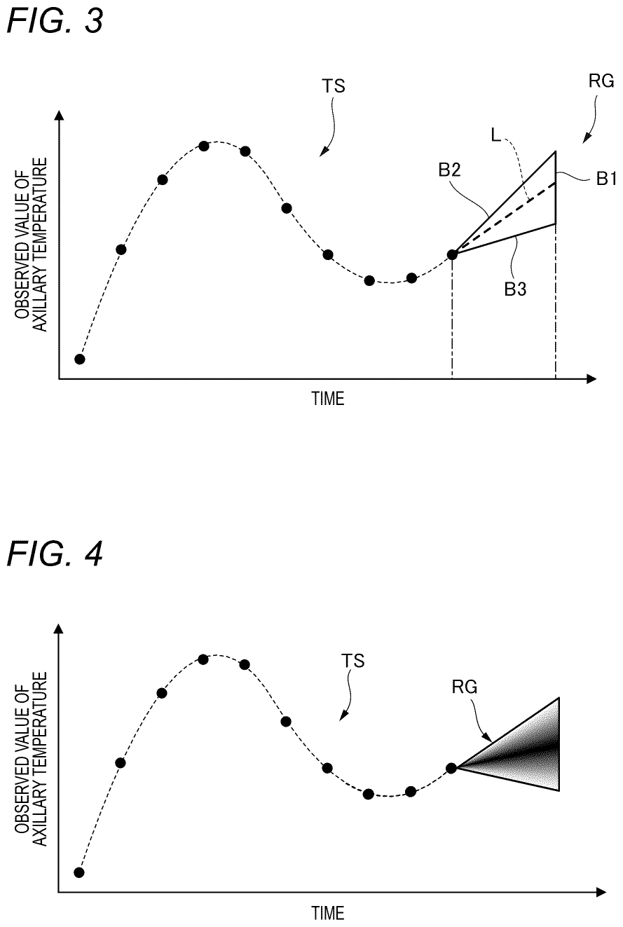

As illustrated in FIG. 3, the range RG within which the unobserved value of the axillary temperature predicted by the prediction model 23 may fall exhibits a triangular shape that is defined by a first boundary line B1, a second boundary line B2, and a third boundary line B3. The first boundary line B1 extends in the vertical direction, and corresponds to a final time point the of a time period in which the forecasting is possible. The second boundary line B2 corresponds to change with time of a possible maximum value of the unobserved value of the axillary temperature from a time point ts, at which the latest observed value included in the time series data TS is acquired, to the time point te. The third boundary line B3 corresponds to change with time of a possible minimum value of the unobserved value of the axillary temperature from the time point ts to the time point te. In other words, the range RG represents change with time of the range within which the unobserved value of the axillary temperature may fall.

The prediction model 23 may be configured to, in addition to the range within which the unobserved value of the axillary temperature may fall, predict change with time of a maximum likelihood value of the unobserved value. The maximum likelihood value is an example of a representative value. In addition to the above-described range RG, the processor 22 may be configured to cause the output device 3 to visualize a line L indicating the change with time of the maximum likelihood value.

In addition to or in place of the maximum likelihood value, other representative values may be subjected to visualization. Examples of other representative values include a mean value, a median value, a quartile, a confidence limit value, and the like.

The prediction model 23 may be configured to predict a probability distribution of an unobserved value at a forecasted time point. In this case, as illustrated in FIG. 4, the processor 22 may be configured to visualize the predicted probability distribution. In this example, the visualization is performed with a color map in which an unobserved value may be acquired with a higher probability corresponds to a darker color. However, the visualization may be performed in a manner such as a violin plot.

At least one of the second boundary line B2 and the third boundary line B3 defining the range RG may include multiple linear lines with different gradients. Alternatively, at least one of the second boundary line B2 and the third boundary line B3 may include a nonlinear portion. Accordingly, the line L to be visualized in the range RG may also include multiple linear portions with different gradients or a non-linear portion in accordance with the shapes of the second boundary line B2 and the third boundary line B3.

As another example, in a case where the time interval of the observed values of the axillary temperature as acquired that are included in the time series data TS exceeds a threshold value, at least one unobserved value of the axillary temperature may be interpolated between the observed values. The threshold value for the time interval may be, for example, the shortest time interval that is assumed in the observation data set used in the above-described machine learning. Namely, the number of interpolated values may be determined such that a time interval between each observed value and an interpolated value adjacent to each other as well as a time interval between adjacent interpolated values (in a case where multiple values are interpolated) coincide with the shortest time interval that is assumed in the observation data set used in the above-described machine learning.

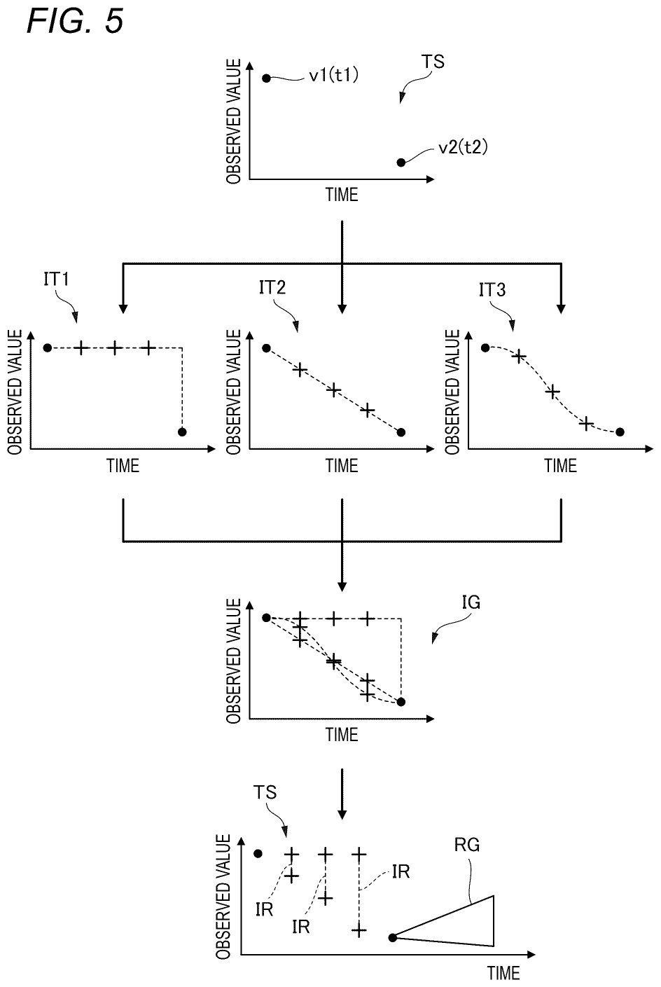

In this case, prediction may be performed with respect to a range within which the interpolated value may fall. The prediction model 23 may be configured to predict a range within which the unobserved value of the axillary temperature may fall based on that range. With reference to FIG. 5, an exemplary specific operation of the prediction model 23 configured as described above will be described. The time series data TS received by the input interface 21 include an observed value v1 of the axillary temperature acquired at a time point t1 and an observed value v2 of the axillary temperature acquired at a time point t2.

The processor 22 is configured to generate multiple interpolation data sets in different manners based on the time series data TS. In this example, a first interpolation data set IT1, a second interpolation data set IT2, and a third interpolation data set IT3 are generated.

In the first interpolation data set IT1, three values of the axillary temperature are interpolated in a time period between the time points t1 and t2. The interpolation method is based on the LOCF (Last Observation Carried Forward) method. In this method, interpolation is performed such that an observed value at a certain time point is maintained until a next observed value is obtained.

In the second interpolation data set IT2, three values of the axillary temperature are interpolated in a time period between the time points t1 and t2. The interpolation method is based on a linear interpolation method. In this method, a value corresponding to an observed value at an arbitrary time point that is located on a linear line connecting two observed values is interpolated.

In the third interpolation data set IT3, three values of the axillary temperature are interpolated in a time period between the time points t1 and t2. The interpolation method is based on a spline interpolation method. In this method, a value corresponding to an observed value at an arbitrary time point that is located on a polynomial curve connecting two observed values is interpolated.

As long as the interpolation methods are different from each other, the number of the multiple interpolation data sets to be generated by the processor 22 may be arbitrarily determined. It should be noted that, as used herein, the expression “different interpolation methods” is not intended to refer to only a case where the types of interpolation methods are different from each other. As an example, an interpolation data set that is generated by spline interpolation using a cubic polynomial, and an interpolation data set that is generated by spline interpolation using a fifth polynomial are interpreted as having the same type of [spline interpolation], but having different interpolation methods. As another example, multiple interpolation data sets generated with the same type of interpolation method but the number of values to be interpolated during the same time period are different from each other are interpreted as “different interpolation methods” as well.

Subsequently, the processor 22 integrates the first interpolation data set IT1, the second interpolation data set IT2, and the third interpolation data set IT3 to generate integrated data IG. In addition, the processor 22 identifies, as a prediction result, a range IR defined by the maximum value and the minimum value among the multiple interpolated values included in the integrated data IG.

The processor 22 determines a specific representative value within the specified range IR as the interpolated value. Examples of the representative value include a maximum value, a minimum value, an average value, an intermediate value, a mode value, and the like. As a result, it is obtained a data set that is to be subjected to the prediction performed by the prediction model 23 generated through the above-described machine learning. The processor 22 predicts, based on the above-described manner, the range RG within which the value of the axillary temperature may fall, and causes the output device 3 to visualize the range RG.

According to the such a configuration, it is still possible to provide the above-described user interface even in a case where it is difficult to obtain such a time series data that includes a sufficient number of observed values of an observed parameter.

Next, another exemplary configuration of the prediction device 2 will be described with reference to FIG. 6. Only the difference from the configuration described with reference to FIGS. 1 to 5 will be described. The above descriptions can be applied to other items.

In this example, the prediction model 23 is configured as a classification model so as to output, in response to the input of the time series data TS including multiple observed values of the axillary temperature acquired from the subject S, a probability of occurrence of an event requiring an antipyretic analgesic agent, as a prediction result. The event that requires an antipyretic analgesic agent is an example of an event related to the observed parameter.

Other examples of the probability of occurrence to be predicted by the classification model may include: a probability of occurrence of an event involving respiratory management with a respirator with respect to an input of time series data including observed values of respiratory rate, heart rate, arterial oxygen saturation or the like that are intermittently observed; a probability of occurrence of an acute renal failure with respect to an input of time series data including urine volumes that are regularly measured; and a probability of death in hospital with respect to an input of time series data including observed values of respiratory rate, heart rate, blood pressure or the like that are intermittently observed.

As the probability of occurrence of an event related to the observed parameter, only a value at a specific time point may be predicted, or time series data including multiple predicted values may be formed as illustrated in FIG. 6. The predicted value of the probability of occurrence may or may not be subjected to visualization processing.

As illustrated in FIG. 6, the processor 22 causes the output device 3 to visualize a range RG within which the probability of occurrence of an event that requires an antipyretic analgesic agent may fall, that is predicted by the prediction model 23. The black circle in the figure indicates a maximum likelihood value of the occurrence probability. The maximum likelihood value is an example of a representative value. Examples of other representative values include a mean value, a median value, a quartile, a confidence limit value, and the like. The visualization of the representative value may be omitted.

The prediction model 23 that performs such an operation is generated by the prediction model generating device 4 in the same manner as the manner described with reference to FIG. 2.

The input interface 41 receives a first observation data set OB1 including multiple observed values of the axillary temperature acquired from a living body with a first time interval T1.

The processor 42 is configured to, based on multiple observed values of the axillary temperature that are acquired from the living body with the first time interval T1, estimate a probability that an event requiring an antipyretic analgesic agent occurred with or after the acquisition.

The processor 42 stores, in association with the first time interval T1, a difference between a value corresponding to whether or not the event requiring an antipyretic analgesic agent actually occurs with or after the acquisition of the first observation data set OB1, and the occurrence probability of the event estimated on the basis of the first observation data set OB1. As an example, a value corresponding to the case where the event occurs is set to 100%, and a value corresponding to the case where the event does not occur is set to 0%. The stored information forms a part of training data TR.

Similarly, the input interface 41 receives a second observation data set OB2 including multiple observed values of the axillary temperature acquired from the living body with the second time interval T2.

The processor 42 performs interpolation such that the time interval T2 of the second observation data set OB2 is resampled with the first time interval T1. The processor 42 then calculates a difference between a value corresponding to whether or not the event requiring an antipyretic analgesic agent actually occurs with or after the acquisition of the second observation data set OB2, and the occurrence probability of the event estimated on the basis of the resampled second observation data set OB2. The processor 42 then stores the difference in association with the second time interval T2. As an example, a value corresponding to the case where the event occurs is set to 100%, and a value corresponding to the case where the event does not occur is set to 0%. The stored information forms a part of training data TR.

Moreover, the input interface 41 receives a third observation data set OB3 including multiple observed values of the axillary temperature acquired from the living body with a third time interval T3.

The processor 42 performs interpolation such that the time interval T3 of the third observation data set OB3 is resampled with the first time interval T1. The processor 42 then calculates a difference between a value corresponding to whether or not the event requiring an antipyretic analgesic agent actually occurs with or after the acquisition of the third observation data set OB3, and the occurrence probability of the event estimated on the basis of the resampled third observation data set OB3. The processor 42 stores the difference in association with the third time interval T3. As an example, a value corresponding to the case where the event occurs is set to 100%, and a value corresponding to the case where the event does not occur is set to 0%. The stored information forms a part of training data TR.

As long as the difference can be defined, the value corresponding to whether or not an event actually occurs, as well as the predicted occurrence probability of the event may have an adequate value other than the percentage.

The same processing may be performed on another observation data set including observed values of the axillary temperature acquired with a different time interval to generate the training data TR.

The processor 42 executes machine learning of the prediction model 23 with the training data TR generated as described above. Specifically, the neural network is caused to learn the influence of the time interval of multiple observed values of the axillary temperature acquired from the subject on the estimation accuracy of the occurrence probability of an event requiring an antipyretic analgesic agent. As a result, the prediction model 23 is configured to predict a range within which an occurrence probability of an event requiring an antipyretic analgesic agent may fall, with the time interval of the multiple observed values included in the inputted time series data TS of the axillary temperature as the feature.

In general, the shorter the time interval of acquired multiple observed values included in the time series data TS of the axillary temperature, the higher the occurrence probability of an event requiring an antipyretic analgesic agent that is estimated by the prediction model 23. On the other hand, there may be a case where it is changed (not constant) the time interval for acquiring multiple observed values of a certain observed parameter like the axillary temperature. In other words, there may be a case where the prediction accuracy of the occurrence probability as estimated dynamically changes. However, according to the configuration of this exemplary embodiment, not only it is possible to visualize a range within which the occurrence probability of the event requiring the antipyretic analgesic agent predicted in response to the inputted time series data TS of the axillary temperature may fall, but also it is possible to dynamically change the range in accordance with the time interval of the acquired observed values. Accordingly, it is possible to provide a user interface that enables the user to visually recognize a change in the estimation accuracy of the occurrence probability that may occur in accordance with a change in the observation environment of the axillary temperature.

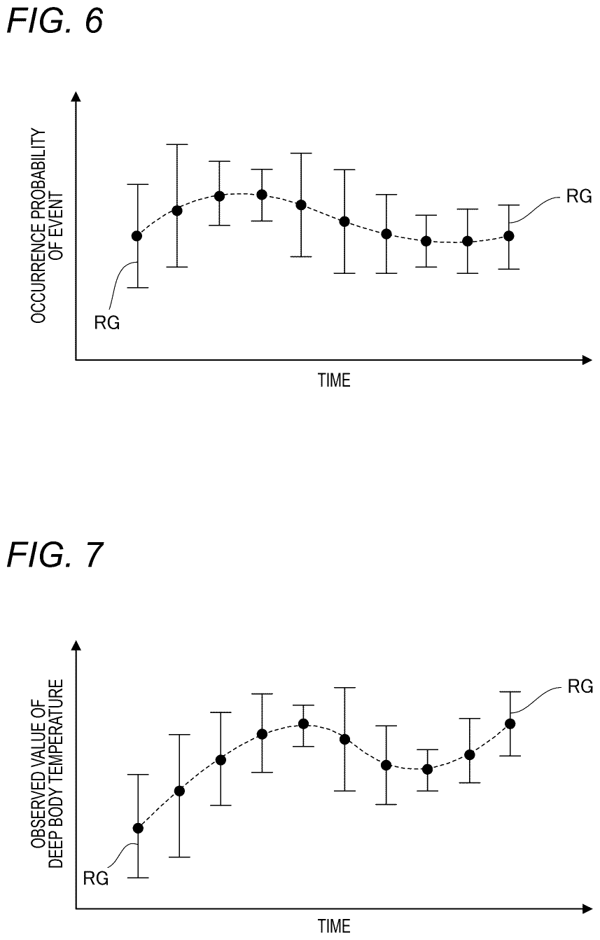

Next, still another exemplary configuration of the prediction device 2 will be described with reference to FIG. 7. Only the difference from the configuration described with reference to FIGS. 1 to 5 will be described. The above descriptions can be applied to other items.

In this example, the prediction model 23 is configured as a regression model so as to output, in response to the input of the time series data TS including multiple observed values of the axillary temperature acquired from the subject S, an observed value of deep body temperature of the subject S, as a prediction result. The deep body temperature is an example of another observed parameter.

Other examples of the observed parameter for which the observed value is predicted with the regression model may include: a cardiac index with respect to an input of time series data including observed values of heart rate, non-invasive blood pressure or the like that are intermittently observed; a blood creatinine with respect to an input of time series data including urine volumes that are regularly measured; and a remaining length of stay in an intensive care unit (ICU) with respect to an input of blood examination results that are regularly performed as well as time series data including observed values of body temperature, respiratory rate, heart rate, blood pressure or the like that are intermittently observed.

As the observed value of another observed parameter, only the value at a specific time point may be predicted, or time series data including multiple estimated values may be formed as illustrated in FIG. 7. The predicted observed value may or may not be subjected to visualization processing.

As illustrated in FIG. 7, the processor 22 causes the output device 3 to visualize a range RG within which the observed value of the deep body temperature may fall, that is predicted by the prediction model 23. The black circle in the figure indicates a maximum likelihood value of the observed value. The maximum likelihood value is an example of a representative value. Examples of other representative values include a mean value, a median value, a quartile, a confidence limit value, and the like. The visualization of the representative value may be omitted.

The prediction model 23 that performs such an operation is generated by the prediction model generating device 4 in the same manner as the manner described with reference to FIG. 2.

The input interface 41 receives a first observation data set OB1 including multiple observed values of the axillary temperature acquired from a living body with a first time interval T1.

The processor 42 is configured to, based on multiple observed values of the axillary temperature that are acquired from the living body with the first time interval T1, estimate an observed value of the deep body temperature acquired with or after the acquisition.

The processor 42 stores, in association with the first time interval T1, a difference between the observed value of the deep body temperature acquired with or subsequently to the first observation data set OB1 and the observed value as estimated on the basis of the first observation data set OB1. The stored information forms a part of training data TR.

Similarly, the input interface 41 receives a second observation data set OB2 including multiple observed values of the axillary temperature acquired from the living body with the second time interval T2.

The processor 42 performs interpolation such that the time interval T2 of the second observation data set OB2 is resampled with the first time interval T1. In addition, the processor 42 calculates a difference between an observed value of the deep body temperature acquired with or subsequently to the second observation data set OB2 and the observed value as estimated on the basis of the resampled second observation data set OB2. The processor 42 then stores the difference in association with the second time interval T2. The stored information forms a part of training data TR.

Moreover, the input interface 41 receives a third observation data set OB3 including multiple observed values of the axillary temperature acquired from the living body with a third time interval T3.

The processor 42 performs interpolation such that the time interval T3 of the third observation data set OB3 is resampled with the first time interval T1. In addition, the processor 42 calculates a difference between an observed value of the deep body temperature acquired with or subsequently to the third observation data set OB3 and the observed value as estimated on the basis of the resampled third observation data set OB3. The processor 42 stores the difference in association with the third time interval T3. The stored information forms a part of training data TR.

The same processing may be performed on another observation data set including observed values of the axillary temperature acquired with a different time interval to generate the training data TR.

The processor 42 executes machine learning of the prediction model 23 with the training data TR generated as described above. Specifically, the neural network is caused to learn the influence of the time interval of multiple observed values of the axillary temperature acquired from the subject on the estimation accuracy of the observed value of the deep body temperature. As a result, the prediction model 23 is configured to predict a range within which an observed value of the deep body temperature may fall, with the time interval of the multiple observed values included in the inputted time series data TS of the axillary temperature as the feature.

In general, the shorter the time interval of acquired multiple observed values included in the time series data TS of the axillary temperature, the higher the estimation accuracy of the observed value of the deep body temperature that is estimated by the prediction model 23. On the other hand, there may be a case where it is changed (not constant) the time interval for acquiring multiple observed values of a certain observed parameter like the axillary temperature. In other words, there may be a case where the prediction accuracy of the observed value as estimated dynamically changes. However, according to the configuration of this exemplary embodiment, not only it is possible to visualize a range within which an observed value of the deep body temperature predicted in response to the inputted time series data TS of the axillary temperature may fall, but also it is possible to dynamically change the range in accordance with the time interval of the acquired observed values. Accordingly, it is possible to provide a user interface that enables the user to visually recognize a change in the estimation accuracy of the observed value of the deep body temperature that may occur in accordance with a change in the observation environment of the axillary temperature.

Each of the processor 22 of the prediction device 2 and the processor 42 of the prediction model generating device 4 having various functions described above may be implemented by one or more non-exclusive microprocessors configured to cooperate with one or more non-exclusive memories. Examples of the non-exclusive microprocessor include a CPU, an MPU, and a GPU. Examples of the general-purpose memory include a ROM and a RAM. In this case, a computer program for executing the above-described processing may be stored in the ROM. The ROM is an example of a non-transitory computer-readable medium having stored a computer program. The general-purpose microprocessor designates at least a part of the computer program stored in the ROM, loads the designated part in the RAM, and executes the above-described processing in cooperation with the RAM. The computer program may be pre-installed in a non-exclusive memory, or may be downloaded from an external server with a communication network, and then installed in the non-exclusive memory. In this case, the external server is an example of a non-transitory computer readable medium having stored a computer program.

Each of the processor 22 and the processor 42 may be implemented by one or more exclusive integrated circuitries capable of executing the above-described computer program. Examples of the exclusive integrated circuitry include a microcontroller, an ASIC, and an FPGA. In this case, the above-described computer program is pre-installed in a memory element included in the dedicated integrated circuit. The memory element is an example of a non-transitory computer-readable medium having stored a computer program. Each of the processor 22 and the processor 42 may also be implemented by a combination of the non-exclusive microprocessor and the exclusive integrated circuitry.

Each of the configurations exemplified above is merely illustrative for facilitating understanding of the presently disclosed subject matter. Each exemplary configuration may be appropriately modified or combined with another exemplary configuration within the scope of the presently disclosed subject matter.

In the above exemplary embodiment, the prediction device 2 and the prediction model generating device 4 are illustrated as devices independent from each other. In this case, the input interface 21 and the output interface 43 are configured as hardware interfaces. However, both devices may be implemented as different functional modules in a single device. In this case, both interfaces may be implemented as software interfaces.

The prediction model 23 need not be installed in the prediction device 2. Although not illustrated, the prediction device 2 may be connected to an external server device so as to be able to communicate with the external server device via a communication network. In this case, the prediction model 23 may be installed in the external server device.

In the above exemplary embodiment, the prediction model generating device 4 generates the prediction model 23 through the machine learning using the neural network. However, the prediction model 23 may be generated through other machine learning algorithms. Examples of such machine learning algorithms include linear regression, logistic regression, decision trees, random forests, support vector machines, and the like.

As used herein, the meaning of the term “prediction” may be interpreted so as not to exclude the meaning of each of the terms “estimation” and “forecasting”.

1. A prediction device, comprising:
an interface configured to receive time series data including multiple observed values of an observed parameter that are acquired at different time points for obtaining physiological information of a subject; and
a processor configured to:
input the time series data into a prediction model to perform prediction of one or more unobserved values of the observed parameter; and
cause an output device to visualize a range within which the one or more unobserved values may fall,
wherein the range is changed in accordance with a time interval between the different time points.

2. A prediction device, comprising:
an interface configured to receive time series data including multiple observed values of an observed parameter that are acquired at different time points for obtaining physiological information of a subject; and
a processor configured to:
input the time series data into a prediction model to perform prediction of one or more occurrence probabilities of an event related to the observed parameter; and
cause an output device to visualize a range within which the one or more occurrent probabilities may fall,
wherein the range is changed in accordance with a time interval between the different time points.

3. A prediction device, comprising:
an interface configured to receive time series data including multiple observed values of a first observed parameter that are acquired at different time points for obtaining physiological information of a subject; and
a processor configured to:
input the time series data into a prediction model to perform prediction of one or more observed values of a second observed parameter that is different from the first observed parameter; and
cause an output device to visualize a range within which the one or more observed values of the second observed parameter may fall,
wherein the range is changed in accordance with a time interval between the different time points.

4. The prediction device according to claim 1,
wherein the processor is configured to cause the output device to visualize a representative value of the range.

5. The prediction device according to claim 1,
wherein the processor is configured to cause the output device to visualize distribution of the unobserved values as predicted.

6. The prediction device according to claim 1,
wherein the prediction model is configured to predict the range based on the time interval as a feature.

7. The prediction device according to claim 1,
wherein the prediction model is configured to predict the range based on a range within which one or more values interpolated in a time period corresponding to the time interval between the multiple observed values may fall.

8. The prediction device according to claim 2,
wherein the processor is configured to cause the output device to visualize a representative value of the range.

9. The prediction device according to claim 2,
wherein the processor is configured to cause the output device to visualize distribution of the occurrence probabilities as predicted.

10. The prediction device according to claim 2,
wherein the prediction model is configured to predict the range based on the time interval as a feature.

11. The prediction device according to claim 2,
wherein the prediction model is configured to predict the range based on a range within which one or more values interpolated in a time period corresponding to the time interval between the multiple observed values may fall.

12. The prediction device according to claim 3,
wherein the processor is configured to cause the output device to visualize a representative value of the range.

13. The prediction device according to claim 3,
wherein the processor is configured to cause the output device to visualize distribution of the observed values of the second observed parameter as predicted.

14. The prediction device according to claim 3,
wherein the prediction model is configured to predict the range based on the time interval as a feature.

15. The prediction device according to claim 3,
wherein the prediction model is configured to predict the range based on a range within which one or more values interpolated in a time period corresponding to the time interval between the multiple observed values of the first observed parameter may fall.

16. A method of generating, with a computing device, the prediction model according to claim 1, comprising:
acquiring a first observation data set including multiple observed values of the observed parameter acquired from a living body with a first time interval;
acquiring a second observation data set including multiple observed values of the observed parameter acquired from the living body with a second time interval that is different from the first time interval;
storing, in association with the first time interval, a difference between an observed value of the observed parameter acquired subsequently to the first observation data set and an unobserved value of the observed parameter forecasted on the basis of the first observation data set;
storing, in association with the second time interval, a difference between an observed value of the observed parameter acquired subsequently to the second observation data set and an unobserved value of the observed parameter forecasted on the basis of the second observation data set; and
performing machine learning of the prediction model so as to enable prediction of a range within which one or more unobserved values of the observed parameter may fall on the basis of a time interval of multiple observed values of the observed parameter acquired from a subject.

17. A method of generating, with a computing device, the prediction model according to claim 2, comprising:
acquiring a first observation data set including multiple observed values of the observed parameter acquired from a living body with a first time interval;
acquiring a second observation data set including multiple observed values of the observed parameter acquired from the living body with a second time interval that is different from the first time interval;
storing, in association with the first time interval, a difference between a value corresponding to whether or not an event related to the observed parameter actually occurred with or subsequently to acquisition of the first observation data set and a value corresponding to an occurrence probability of the event estimated on the basis of the first observation data set;
storing, in association with the first time interval, a difference between a value corresponding to whether or not the event actually occurred with or subsequently to acquisition of the second observation data set and a value corresponding to an occurrence probability of the event estimated on the basis of the second observation data set; and
performing machine learning of the prediction model so as to enable prediction of a range within which one or more occurrence probabilities of the event may fall on the basis of a time interval of multiple observed values of the observed parameter acquired from a subject.

18. A method of generating, with a computing device, the prediction model according to claim 3, comprising:
acquiring a first observation data set including multiple observed values of the first observed parameter acquired from a living body with a first time interval;
acquiring a second observation data set including multiple observed values of the first observed parameter acquired from the living body with a second time interval that is different from the first time interval;
storing, in association with the first time interval, a difference between an observed value of the second observed parameter acquired subsequently to the first observation data set and an observed value of the second observed parameter estimated on the basis of the first observation data set;
storing, in association with the second time interval, a difference between an observed value of the second observed parameter acquired subsequently to the second observation data set and an observed value of the second observed parameter estimated on the basis of the second observation data set; and
performing machine learning of the prediction model so as to enable prediction of a range within which one or more observed values of the second observed parameter may fall on the basis of a time interval of multiple observed values of the first observed parameter acquired from a subject.

19. The method according to claim 16,
wherein the second observation data set is generated by downsampling one or more of the multiple observed values included in the first observation data set.

20. The method according to claim 17,
wherein the second observation data set is generated by downsampling one or more of the multiple observed values included in the first observation data set.

21. The method according to claim 18,
wherein the second observation data set is generated by downsampling one or more of the multiple observed values included in the first observation data set.

22. A computing device configured to generate the prediction model according to claim 1, comprising:
an interface configured to receive:
a first observation data set including multiple observed values of the observed parameter acquired from a living body with a first time interval; and
a second observation data set including multiple observed values of the observed parameter acquired from the living body with a second time interval that is different from the first time interval; and
a processor configured to:
store, in association with the first time interval, a difference between an observed value of the observed parameter acquired subsequently to the first observation data set and an unobserved value of the observed parameter forecasted on the basis of the first observation data set;
store, in association with the second time interval, a difference between an observed value of the observed parameter acquired subsequently to the second observation data set and an unobserved value of the observed parameter forecasted on the basis of the second observation data set; and
perform machine learning of the prediction model so as to enable prediction of a range within which one or more unobserved values of the observed parameter may fall on the basis of a time interval of multiple observed values of the observed parameter acquired from a subject.

23. A computing device configured to generate the prediction model according to claim 2, comprising:
an interface configured to receive:
a first observation data set including multiple observed values of the observed parameter acquired from a living body with a first time interval; and
a second observation data set including multiple observed values of the observed parameter acquired from the living body with a second time interval that is different from the first time interval; and
a processor configured to:
store, in association with the first time interval, a difference between a value corresponding to whether or not an event related to the observed parameter is actually occurred with or subsequently to acquisition of the first observation data set and a value corresponding to an occurrence probability of the event estimated on the basis of the first observation data set;
store, in association with the first time interval, a difference between a value corresponding to whether or not the event is actually occurred with or subsequently to acquisition of the second observation data set and a value corresponding to an occurrence probability of the event estimated on the basis of the second observation data set; and
perform machine learning of the prediction model so as to enable prediction of a range within which one or more occurrence probabilities of the event may fall on the basis of a time interval of multiple observed values of the observed parameter acquired from a subject.

24. A computing device configured to generate the prediction model according to claim 3, comprising:
an interface configured to receive:
a first observation data set including multiple observed values of the first observed parameter acquired from a living body with a first time interval; and
a second observation data set including multiple observed values of the first observed parameter acquired from the living body with a second time interval that is different from the first time interval; and
a processor configured to:
store, in association with the first time interval, a difference between an observed value of the second observed parameter acquired subsequently to the first observation data set and an observed value of the second observed parameter estimated on the basis of the first observation data set;
store, in association with the second time interval, a difference between an observed value of the second observed parameter acquired subsequently to the second observation data set and an observed value of the second observed parameter estimated on the basis of the second observation data set; and
perform machine learning of the prediction model so as to enable prediction of a range within which one or more observed values of the second observed parameter may fall on the basis of a time interval of multiple observed values of the first observed parameter acquired from a subject.
